(19) United States
(12) Patent Application Publication
(10) Pub. No.: US 2013/0035506 A1
(43) Pub. Date: Feb. 7, 2013

(54) PROCESS FOR PRODUCING SULFUR-CONTAINING AMINO ACIDS

(71) Applicant: Koji Hagiya, Ibaraki-shi (JP)

(72) Inventor: Koji Hagiya, Ibaraki-shi (JP)

(73) Assignee: SUMITOMO CHEMICAL COMPANY, LIMITED, Chuo-ku, Tokyo (JP)

(21) Appl. No.: 13/639,217

(22) PCT Filed: Apr. 5, 2011
(86) PCT No.: PCT/JP2011/058958

§ 371 Date: Oct. 4, 2012

(30) Foreign Application Priority Data
Apr. 6, 2010 (JP) 2010-087563
Feb. 16, 2011 (JP) 2011-030606

(51) Int. Cl. C07C 319/20

(52) U.S. Cl. 562/526

(57) ABSTRACT

The present invention relates to a process for producing a sulfur-containing amino acid, comprising a step of oxidizing a 2-aminoethanol compound having, at position 2, a sulfur-containing hydrocarbon group having 1 to 24 carbon atoms in the presence of oxygen and at least one transition metal selected from the group consisting of the elements of Groups 8, 9 and 10 of the periodic table.

### TECHNICAL FIELD

The present application is filed, claiming the priorities based on the Japanese Patent Application Nos. 2010-087563 (filed on Apr. 6, 2010) and 2011-030606 (filed on Feb. 16, 2011), and a whole of the contents of the applications is incorporated herein by reference.

The present invention relates to a process for producing a sulfur-containing amino acid.

### BACKGROUND ART

Sulfur-containing amino acids such as methionine and S-alkyl cysteine exist commonly in the all organisms, and they are useful components for many important biological reactions. Particularly, methionine is an essential amino acid, which is an important compound for use as a feed additive.

For example, the following method is disclosed in “industrial organic chemistry”, Tokyo Kagaku-Dojin, 1978, pp. 273-275: 3-(methylthio)propionaldehyde obtained by addition of methanethiol to acrolein is reacted with hydrogen cyanide to obtain 2-hydroxyl-4-methylthiobutyronitrile; and then, the 2-hydroxyl-4-methylthiobutyronitrile is reacted with ammonium carbonate to obtain a substituted hydantoin and thereafter, the substituted hydantoin is hydrolyzed with an alkali. In addition, the following method is disclosed in “Chem. Ber.”, vol. 121, 1988, pp. 2209-2223: methanethiol is added to methyl 2-chloroacrylate; and then, the resultant adduct is reacted with a sodium azide and thereafter, the resultant product is hydrogenated under acidic conditions.

### DISCLOSURE OF INVENTION

### Problem to be Solved by the Invention

However, the methods disclosed in the above documents require using hydrogen cyanide or sodium azide as a raw material. However, these compounds require careful handling.

Under such a circumstance, there has been demanded a new process for producing sulfur-containing amino acids without using of hydrogen cyanide or sodium azide.

### Means for Solving the Problem

As a result of the present inventors' intensive studies for solving the above-described problem, the present invention is accomplished.

The present invention provides the followings:
[1] A process for producing a sulfur-containing amino acid, comprising a step of oxidizing a 2-aminoethanol compound having, at position 2, a sulfur-containing hydrocarbon group having 1 to 24 carbon atoms in the presence of oxygen and at least one transition metal selected from the group consisting of the elements of Groups 8, 9 and 10 of the periodic table.
[2] The process according to the above item [1], wherein the above-described step of oxidizing the 2-aminoethanol compound is carried out further in the presence of at least one typical metal compound selected from the group consisting of alkali metal compounds and alkaline earth metal compounds.
[3] The process according to the above item [2], wherein the above-described typical metal compound is at least one compound selected from the group consisting of alkali metal hydroxides and alkali metal carbonates.
[4] The process according to any one of the above items [1] to [3], wherein the above-described step of oxidizing the 2-aminoethanol compound is carried out further in the presence of a solvent.
[5] The process according to any one of the above items [1] to [4], wherein the above-described transition metal is at least one metal selected from the group consisting of platinum group elements.
[6] The process according to any one of the above items [1] to [5], wherein the above-described sulfur-containing hydrocarbon group has no multiple bond.
[7] The process according to any one of the above items [1] to [6], wherein the above-described 2-aminoethanol compound is 2-amino-4-methylthio-1-butanol.

According to the present invention, a new process for producing the sulfur-containing amino acids without using as a raw material any hydrogen cyanide or sodium azide which requires careful handling can be provided.

### MODES FOR CARRYING OUT THE INVENTION

Hereinafter, the present invention will be described in detail.

The 2-aminoethanol compound has a sulfur-containing hydrocarbon group at position 2 (the 2-aminoethanol compound is sometimes referred to as “alcohol compound”), for example, which is represented by the following formula:

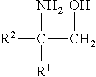

In the formula, R² and R² independently represent a sulfur-containing hydrocarbon group or a hydrogen atom, one of which represents the sulfur-containing hydrocarbon group. Herein, the sulfur-containing hydrocarbon group means a group comprising a sulfur atom, a carbon atom and a hydrogen atom. The hydrogen atom in the sulfur-containing hydrocarbon group may be substituted by a group inactive to an oxidation reaction as will be described later.

There is no limit in selection of the sulfur-containing hydrocarbon group if the group has 1 to 24 carbon atoms. The group may be a saturated sulfur-containing hydrocarbon group having no multiple bond, or a unsaturated sulfur-containing hydrocarbon group having a double bond and/or a triple bond. The unsaturated sulfur-containing hydrocarbon group may contain an aromatic isocyclic ring such as a benzene ring and/or an aromatic heterocyclic ring such as a thiophene ring.

The saturated sulfur-containing hydrocarbon group may be linear, branched or cyclic. Hereinafter, the linear or branched saturated sulfur-containing hydrocarbon group is sometimes referred to as a saturated chain sulfur-containing hydrocarbon group. The cyclic saturated sulfur-containing hydrocarbon group is sometimes referred to as a saturated cyclic sulfur-containing hydrocarbon group.

The saturated chain sulfur-containing hydrocarbon group includes a methylthiomethyl group, an ethylthiomethyl group, a propylthiomethyl group, an isopropylthiomethyl group, a tert-butylthiomethyl group, a 1-(methylthio)ethyl group, a 2-(methylthio)ethyl group, a 1-(ethylthio)ethyl group, a 2-(ethylthio)ethyl group, a 1-(propylthio)ethyl group, a 2-(propylthio)ethyl group, a 2-(isopropylthio)ethyl group, a 2-(tert-butylthio)ethyl group, a 1-(methylthio)propyl group, a 2-(methylthio)propyl group, a 3-(methylthio)propyl group, a 3-(ethylthio)propyl group, a 3-(propylthio)propyl group, a 3-(isopropylthio)propyl group and a 2,3-(dimethylthio)propyl group.

The saturated cyclic sulfur-containing hydrocarbon groups include a cyclopropylthiomethyl group, a cyclobutylthiomethyl group, a cyclopentylthiomethyl group, a cyclohexylthiomethyl group, a 2-(methylthio)cyclopropyl group, a 2-(methylthio)cyclobutyl group, a 2-(methylthio)cyclopentyl group, a 2-(methothio)cyclohexyl group, a 4-(methylthio)cyclohexyl group, a 2-methyl-4-(methylthio)cyclohexyl group, a 2,4-(dimethylthio)cyclohexyl group, a 2-thiacyclohexyl group and 4-thiacyclohexyl group.

The unsaturated sulfur-containing hydrocarbon group includes a vinylthiomethyl group, a 1-(vinylthio)ethyl group, a 2-(vinylthio)ethyl group, a 4-methylthio-1-butenyl group, a 4-methylthio-2-butenyl group, a 2-methylthiophenyl group, a 3-methylthiophenyl group, a 4-methylthiophenyl group, a 2-methyl-4-methylthiophenyl group, a 2,4-(dimethylthio)phenyl group, a phenylthiomethyl group, a 1-(phenylthio)ethyl group, a 2-(phenylthio)ethyl group, a benzylthiomethyl group, a 1-(benzylthio)ethyl group, a 2-(benzylthio)ethyl group, a 2-thienyl group, a 3-thienyl group and a 2-methyl-3-thienyl group.

The group inactive to an oxidation reaction includes C₁₋₁₂ alkyloxy groups such as a methoxy group, an ethoxy group, a propyloxy group, an isopropyloxy group, a butyloxy group, an isobutyloxy group, a sec-butyloxy group, a tert-butyloxy group, a pentyloxy group and a hexyloxy group;
C₇₋₁₂ aralkyloxy groups such as a benzyl group;
C₃₋₈ cycloalkyloxy groups such as a cyclopropyloxy group, a cyclobutyloxy group, a cyclopentyloxy group and a cyclohexyloxy group;
C₆₋₁₂ aryloxy groups such as a phenoxy group, a 2-methylphenoxy group, a 4-methylphenoxy group and a 4-phenylphenoxy group;
C₁-₆ perfluoroalkyloxy groups such as a trifluoromethoxy group and a pentafluoroethoxy group;
substituted or unsubstituted amino groups, among which the substituted amino group has usually 1 to 12 carbon atoms, such as an amino group, a methylamino group, a dimethylamino group, a benzylamino group, a tert-butoxycarbonylamino group and a benzyloxycarbonylamino group;
C₂₋₁₂ acyl groups such as an acetyl group, a propionyl group, a butylyl group, an isobutylyl group, a valeryl group, an isovaleryl group, a pivaloyl group and a benzoyl group;
C₂₋₁₂ acyloxy groups such as an acetyloxy group, a propionyloxy group, a butylyloxy group, an isobutylyloxy group, a valeryloxy group, an isovaleryloxy group, a pivaloyloxy group and a benzoyloxy group; and
halogen atoms such as a fluorine atom and a chlorine atom.

The hydrogen groups of C₆₋₁₂ aryloxy groups and C₇₋₁₂ aralkyloxy groups may be substituted by at least one selected from the group consisting of C₁₋₁₂ alkyloxy groups, C₆₋₁₂ aryloxy groups, halogen atoms and the like.

The sulfur-containing hydrocarbon group is preferably a saturated sulfur-containing hydrocarbon group having no multiple bond, more preferably a saturated chain sulfur-containing hydrocarbon group, still more preferably a 2-(C₁₋₁₂ alkylthio) (C₁₋₆ alkyl) group, particularly preferably a 2-(methylthio)ethyl group.

The alcohol compound includes specifically 2-amino-3-methylthio-1-propanol, 2-amino-3-tert-butylthio-1-propanol, 2-amino-3-benzylthio-1-propanol, 2-amino-3-ethylthio-1-propanol, 2-amino-4-methylthio-1-butanol, 2-amino-4-ethylthio-1-butanol, 2-amino-4-propylthio-1-butanol, 2-amino-4-benzylthio-1-butanol, 2-amino-5-methylthio-1-pentanol, 2-amino-5-ethylthio-1-pentanol, 2-amino-5-propylthio-1-pentanol, 2-amino-6-butylthio-1-heptanol and 2-amino-5-benzylthio-1-pentanol, preferably 2-amino-4-methylthio-1-butanol.

As the alcohol, a commercially available product may be used, and also, the alcohol produced by using any known method such as a method by reacting an ethylene oxide having a sulfur-containing hydrocarbon group with ammonia (e.g., Izvestiya Akademii Nauk SSSR, Seriya Khimicheskaya, vol. 9, pp 2090-2094, 1985) or the like.

The alcohol compound is oxidized in the presence of at least one transition metal (hereinafter sometimes referred to as a transition metal catalyst) selected from the group of consisting of the elements of Groups 8, 9 and 10 of the periodic table. Hereinafter, the reaction of oxidizing the alcohol compound in the presence of the transition metal catalyst and oxygen is sometimes referred to as an oxidation reaction or the present reaction. The alcohol compound is converted to a sulfur-containing amino acid by the present reaction.

The elements of Group 8 of the periodic table include iron, ruthenium. The elements of Group 9 of the periodic table include cobalt, rhodium. The elements of Group 10 of the periodic table include nickel, palladium, platinum. The transition metal is preferably at least one metal selected from platinum group elements, more preferably ruthenium or platinum, still more preferably platinum.

The transition metal catalyst may be supported on a support (hereinafter, the transition metal catalyst supported on the support is sometimes referred to as a supported catalyst) or may not be supported thereon. Also, the transition metal catalyst may be a catalyst in which an alloy containing at least one transition metal selected from the group of consisting of the elements of Groups 8, 9 and 10 of the periodic table is treated with an acid or an alkali (hereinafter sometimes referred to as a developing catalyst).

The support includes at least one selected from the group consisting of an activated carbon, alumina, silica, zeolite, diatomaceous earth and zirconium oxide. It is preferable that the support has a larger surface area in order to improve reactivity. The supported catalyst may be commercially available product, or may be a catalyst obtained as follows: at least one transition metal selected from the group of consisting of the elements of Groups 8, 9 and 10 of the periodic table, or an alloy of such a transition metal with aluminum, is supported on the above-described support to obtain the supported catalyst. Otherwise, the supported catalyst may be a catalyst obtained as follows: at least one salt selected from the group consisting of nitrates, sulfates, formates, acetates, carbonates, halides, hydroxides and oxides of these transition metals is supported on the above-described support by coprecipitation process or impregnation process, and then this supported salt is reduced with hydrogen or is calcined.

The transition metal catalyst is preferably a developing catalyst or a supported catalyst, more preferably a supported catalyst.

The amount of the transition metal catalyst to be used may vary depending on the form of the transition metal catalyst in use, and is preferably 0.001 mole or more per mole of the alcohol compound. When the transition metal catalyst is a supported catalyst, the amount of the catalyst including the support is usually from 0.1 to 100 parts by weight per part by the weight of the alcohol compound. The amount of the transition metal catalyst to be used is preferably 0.5 mole or less per mole of the alcohol compound from an economical viewpoint.

The oxygen may be an oxygen gas, or an oxygen gas diluted with an inert gas such as a nitrogen gas, or oxygen in an air. The oxygen in an air may be diluted with an inert gas such as a nitrogen gas, for use.

The amount of the oxygen to be used is preferably one mole or more per mole of the alcohol compound. The upper limit is not limited, but it is usually 100 moles per mole of the alcohol compound.

Preferably, the present reaction is carried out further in the presence of at least one typical metal compound selected from the group consisting of alkali metal compounds and alkaline earth metal compounds.

Examples of the alkali metal compounds include alkali metal carbonates such as sodium carbonate, sodium bicarbonate, potassium carbonate, potassium bicarbonate, lithium carbonate and lithium bicarbonate; and alkali metal hydroxides such as sodium hydroxide, potassium hydroxide and lithium hydroxide.

Examples of the alkaline earth metal compounds include alkaline earth metal carbonates such as magnesium carbonate and calcium carbonate; and alkaline earth metal hydroxides such as magnesium hydroxide and calcium hydroxide.

The typical metal compound is preferably at least one metal selected from the group consisting of typical metal hydroxides and typical metal carbonates, more preferably at least one metal selected from the group consisting of alkali metal hydroxides and alkali metal carbonates, still more preferably sodium hydroxide or sodium bicarbonate, particularly preferably sodium hydroxide.

The amount of the typical metal compound to be used is preferably one mole or more per mole of the alcohol compound, while the upper limit thereof is not limited. The amount of the typical metal compound to be used is 2 moles or less per mole of the alcohol compound from a practical viewpoint.

Preferably, the present reaction is carried out further in the presence of a solvent.

There is no limit in selection of the solvent if it does not hinder the present reaction. Examples of the solvent include ester solvents such as ethyl acetate; nitrile solvents such as acetonitrile and propionitrile; water; and mixtures thereof. The solvent is preferably water, a mixture of water with an ester solvent or a mixture of water with a nitrile solvent, more preferably a mixture of water with a nitrile solvent, still more preferably a mixture of water with acetonitrile.

The amount of the solvent to be used is, which is not limited, practically 100 parts by weight or less per one part by weight of the alcohol compound.

In the present reaction, the order of blending the reactants is not limited. For example, in a preferred mode, the alcohol compound, the transition metal catalyst, the typical metal compound and the solvent are mixed, and then, the resulting mixture is mixed with oxygen.

The present reaction may be carried out under reduced pressure or normal pressure or increased pressure. Preferably, the present reaction is carried out under normal pressure or increased pressure.

A temperature for the present reaction may vary depending on an amount of the transition metal catalyst to be used, an amount of oxygen to be used, etc., and is preferably from 0 to 150° C., more preferably from 20 to 100° C. A reaction temperature not lower than 0° C. tends to permit a higher rate of the oxidation reaction. A reaction temperature not higher than 150° C. tends to higher selectivity for the oxidation reaction.

The reaction time may vary depending on the reaction temperature, the reactants to be used or the like, and is, for example, from 0.5 to 50 hours.

The degree of the present reaction progress can be confirmed by analytic means such as gas chromatography, high-performance liquid chromatography, thin-layer chromatography, nuclear magnetic resonance spectroscopy, infrared absorption spectroscopy or the like.

After completion of the reaction, the sulfur-containing amino acid may be brought out by a procedure in which the resultant reaction mixture is filtered to remove the transition metal catalyst therefrom, and then, the reaction mixture is optionally neutralized with mineral acid such as sulfuric acid or hydrochloric acid and is then concentrated and cooled. If the sulfur-containing amino acid is a lipophilic compound, the sulfur-containing amino acid may be brought out by a procedure in which the resultant reaction mixture is filtered to remove the transition metal catalyst and is then mixed with a solvent immiscible to water, and the resultant mixture is extracted, concentrated and cooled. The solvent immiscible to water includes ester solvents such as ethyl acetate, and ether solvents such as methyl tert-butyl ether. The amount of the immiscible solvent to be used is not limited.

The sulfur-containing amino acid thus brought out may be purified by distillation, column chromatography, crystallization or the like.

The sulfur-containing amino acid thus obtained is α-amino acid having the sulfur-containing hydrocarbon group at position 2.

The sulfur-containing amino acid is preferably represented as follow:

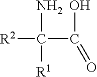

(In the formula, R¹ and R² are defined as above.)

Examples of such an amino acid include 2-amino-3-(methylthio)propionic acid, 2-amino-3-(tert-butylthio)propionic acid, 2-amino-3-(benzylthio)propionic acid, 2-amino-3-(ethylthio)propionic acid, 2-amino-4-(methylthio)butyric acid (i.e., methionine), 2-amino-4-(ethylthio)butyric acid, 2-amino-4-(propylthio)butyric acid, 2-amino-4-(benzylthio)butyric acid, 2-amino-5-(methylthio)pentanoic acid, 2-amino-3-(ethylthio)pentanoic acid, 2-amino-3-(propylthio)pentanoic acid and 2-amino-3-(benzylthio)pentanoic acid.

### EXAMPLES

Hereinafter, the present invention will be described in more detail by way of Examples.

### Example 1

### Production of 2-amino-4-(methylthio)butyric acid

A 50-mL pressure reaction tube equipped with a magnetic rotor was charged with 2-amino-4-methylthio-1-butanol (135 mg), sodium hydroxide (40 mg), water (1 g), acetonitrile (1 g) and a 5 wt. % Pt/C (containing 50% by weight of water) (100 mg), and the interior of the reaction tube was compressed with an air up to 1 MPa. The resulting mixture was stirred at 50° C. for 8 hours. The reaction mixture was cooled to room temperature and was then filtered. The resulting filtrate was neutralized with 0.1N sulfuric acid, and the solvent was distilled off to obtain 2-amino-4-(methylthio)butyric acid.

### Determination of Yield

Methanol (5 g) was added to the resultant 2-amino-4-(methylthio)butyric acid, and a 10 wt. % hexane solution of trimethylsilyldiazomethane was further added thereto, to obtain methyl 2-amino-4-(methylthio)butyrate. A methanol solution containing the resultant methyl 2-amino-4-(methylthio)butyrate was analyzed by a gas chromatography internal standard method to determine a yield of methyl 2-amino-4-(methylthio)butyrate from 4-(methylthio)-2-amino-1-butanol. As a result, the yield was 14%. In other words, 2-amino-4-(methylthio)butyric acid was obtained at a yield of 14% or more from 2-amino-4-methylthio-1-butanol. 80% of 2-amino-4-methylthio-1-butanol used as the starting material was recovered.

### Example 2

### Production of 2-amino-4-(methylthio)butyric acid

A 50-mL flask equipped with a magnetic rotor was charged with 2-amino-4-methylthio-1-butanol (100 mg), sodium bicarbonate (70 mg), acetonitrile (3 g) and a 5 wt. % Pt/C (containing 50% by weight of water) (100 mg), and the resulting mixture was stirred at 60° C. for 8 hours under an atmosphere of an air. The reaction mixture was cooled to room temperature and was then filtered. The resulting filtrate was neutralized with 0.1N sulfuric acid, and the solvent was distilled off from the mixture to obtain 2-amino-4-(methylthio)butyric acid.

### Determination of Yield

Methanol (5 g) was added to the resultant 2-amino-4-(methylthio)butyric acid, and a 10 wt. % hexane solution of trimethylsilyldiazomethane was further added thereto, to obtain methyl 2-amino-4-(methylthio)butyrate. A methanol solution containing the resultant methyl 2-amino-4-(methylthio)butyrate was analyzed by a gas chromatography internal standard method to determine the yield of methyl 2-amino-4-(methylthio)butyrate from 4-(methylthio)-2-amino-1-butanol. As a result, the yield was 9%. In other words, 2-amino-4-(methylthio)butyric acid was obtained at a yield of 9% or more from 2-amino-4-methylthio-1-butanol. 90% of 2-amino-4-methylthio-l-butanol used as the starting material was recovered.

### Example 3

### Production of 2-amino-4-(methylthio)butyric acid

A 50-mL flask equipped with a magnetic rotor was charged with 2-amino-4-methylthio-1-butanol (100 mg), sodium bicarbonate (30 mg), water (1 g), acetonitrile (1 g) and a 5 wt. % Ru/C (containing 50% by weight of water) (50 mg), and the resulting mixture was stirred at 50° C. for 8 hours under an atmosphere of an air. The reaction mixture was cooled to room temperature and was then filtered. The resulting filtrate was neutralized with 0.1N sulfuric acid, and the solvent was distilled off to obtain 2-amino-4-(methylthio)butyric acid.

### Determination of Yield

Methanol (5 g) was added to the resultant 2-amino-4-(methylthio)butyric acid, and a 10 wt. % hexane solution of trimethylsilyldiazomethane was further added thereto, to obtain methyl 2-amino-4-(methylthio)butyrate. A methanol solution containing the resultant methyl 2-amino-4-(methylthio)butyrate was analyzed by a gas chromatography internal standard method to determine the yield of methyl 2-amino-4-(methylthio)butyrate from 4-(methylthio)-2-amino-1-butanol. As a result, the yield was 5%. In other words, 2-amino-4-(methylthio)butyric acid was obtained at a yield of 5% or more from 2-amino-4-methylthio-1-butanol. 90% of 2-amino-4-methylthio-1-butanol used as the starting material was recovered.

### INDUSTRIAL APPLICABILITY

The present invention is industrially applicable as a process for producing the sulfur-containing amino acids such as methionine.

1. A process for producing a sulfur-containing amino acid, comprising a step of oxidizing a 2-aminoethanol compound having, at position 2, a sulfur-containing hydrocarbon group having 1 to 24 carbon atoms in the presence of oxygen and at least one transition metal selected from the group consisting of the elements of Groups 8, 9 and 10 of the periodic table.

2. The process according to claim 1, wherein the step of oxidizing the 2-aminoethanol compound is carried out further in the presence of at least one typical metal compound selected from the group consisting of alkali metal compounds and alkaline earth metal compounds.

3. The process according to claim 2, wherein the typical metal compound is at least one compound selected from the group consisting of alkali metal hydroxides and alkali metal carbonates.

4. The process according to claim 1, wherein the step of oxidizing the 2-aminoethanol compound is carried out further in the presence of a solvent.

5. The process according to claim 1, wherein the transition metal is at least one metal selected from the group consisting of platinum group elements.

6. The process according to claim 1, wherein the sulfur-containing hydrocarbon group has no multiple bond.

7. The process according to claim 1, wherein the 2-aminoethanol compound is 2-amino-4-methylthio-1-butanol.
